(19) United States
(12) Patent Application Publication
(10) Pub. No.: US 2024/0050237 A1
(43) Pub. Date: Feb. 15, 2024

(54) Augmentable Expanding Implant

(71) Applicant: K2M, Inc., Leesburg, VA (US)

(72) Inventor: Larry E. McClintock, Gore, VA (US)

(73) Assignee: K2M, Inc., Leesburg, VA (US)

(21) Appl. No.: 18/265,321

(22) PCT Filed: Dec. 8, 2021
(86) PCT No.: PCT/US2021/062402

§ 371 Date: Jun. 5, 2023

Related U.S. Application Data
(60) Provisional application No. 63/126,147, filed on Dec. 16, 2020.

(51) Int. Cl. A61F 2/44; A61F 2/46

(52) U.S. Cl. CPC A61F 2/4455; A61F 2/4611; A61F 2002/30537

(57) ABSTRACT

The spinal implant described in the present disclosure utilizes an augmented expanding system to build the desired height spacer in-situ without the need for mechanically expanding devices. An expanding spacer system includes a plurality of implant components configured to mate with each other in the intervertebral disc space and a positioning system used to insert each of the plurality of implant components. Each implant component has a smaller dimension and a larger dimension. The positioning system is used to insert the first component into the disc space with the smaller dimension parallel to the spinal axis, and the positioning system is then used to rotate the first implant component to distract the disc space. Additional implant components are added and rotated to further distract the disc space until a desired height is reached.

### CROSS-REFERENCE TO RELATED APPLICATIONS

This application claims the benefit of the filing date of U.S. Provisional Application No. 63/126,147 filed Dec. 16, 2020, the disclosure of which is hereby incorporated by reference herein in its entirety.

### BACKGROUND OF THE INVENTION

Intervertebral implants are commonly used in spinal surgery, such as in interbody fusion procedures, in which an implant (e.g., a spacer or cage) is placed in the disc space between two vertebrae to be fused together. At least a portion of the disc is typically removed before the implant is positioned in the intervertebral space, and the implant may be supplemented with bone graft material to promote fusion of the vertebrae. Interbody fusion procedures may also be performed in conjunction with other types of fixation, such as pedicle screw fixation, to provide additional stability, particularly while the vertebrae fuse together.

Different interbody fusion procedures can be distinguished by their location along the spine (e.g., in the cervical, thoracic, or lumbar regions); by the type of implant used; and by the surgical approach to the intervertebral space, in which different surgical approaches often imply different structural characteristics of the implant or implants used. Different surgical approaches to the spine include anterior, posterior, and lateral. Examples of interbody fusion techniques performed along a posterior approach include posterior lumbar interbody fusion (PLIF) and transforaminal lumbar interbody fusion (TLIF). PLIF techniques typically include positioning two intervertebral implants into the intervertebral space along a posterior to anterior direction, with one implant being positioned towards the left side of the spine and one implant being positioned towards the right side of the spine. The implants used in such PLIF techniques typically have a straight shape, in that they extend along a central axis. TLIF techniques, by contrast, typically include positioning one intervertebral implant into the intervertebral space (often towards the anterior portion of the intervertebral space) from the posterior of the patient, but the spine is approached on one side from a more lateral position than in PLIF techniques. The implants used in such TLIF techniques are often curved, such that they have an overall kidney bean-like shape. Interbody fusion techniques performed along a lateral approach, on the other hand, often involve implants that are generally symmetric along their linear longitudinal axis (e.g., having a substantially rectangular or oval shape), but the implants are typically larger than those used in PLIF or TLIF techniques. That is, intervertebral implants used in lateral approaches often cover a substantial portion of the disc space.

Included among the different types of intervertebral implants are expandable and static implants. Expandable implants often have an initially contracted configuration, such that they have a low profile in the superior-inferior direction, in order to ease insertion into the intervertebral space. In that regard, vertebral body endplates tend to have a slightly concave shape, such that the central portion of the disc space generally defines a larger intervertebral distance than the outer perimeter (known as the apophyseal ring). Expandable implants often include a mechanism that can drive top and bottom portions of the implant apart to expand the implant in the superior-inferior direction after the implant is positioned in the intervertebral space past the apophyseal ring, so as to securely engage and stabilize the vertebrae on both sides of the intervertebral space. Static implants may be non-expandable. Insertion of static implants may be more traumatic than that of expandable implants. Insertion of static implants may involve hammering the implant in the desired spacer size to get it past the apophyseal ring into the disc space, particularly for static implants that are sized for the dimension of the intervertebral space towards the central portion of the disc space.

Although considerable effort has been devoted in the art of optimization of such expandable and static implants, further improvement would be desirable.

### BRIEF SUMMARY OF THE INVENTION

The spinal implant described in the present disclosure utilizes an augmented expanding system that enables a surgeon to build the desired height spacer in-situ without the need for mechanically expanding devices. An expanding spacer system may include a plurality of implant components configured to mate with each other in the intervertebral disc space and a positioning system used to insert each of the plurality of implant components. Each implant component may have a smaller dimension and a larger dimension. The positioning system may be used to insert the first component into the disc space with the smaller dimension parallel to the spinal axis, and a device (e.g., the positioning system) may then be used to rotate the first implant component so that the larger dimension is parallel to the spinal axis, thereby distracting the disc space. A second implant component may be inserted around the first component using the positioning system such that the smaller dimension of the outer, second component aligns with the larger dimension of the inner, first component, the smaller dimension of the outer component being parallel to the spinal axis. Both components may then be rotated by a device (e.g., the positioning system) so that the larger dimension of the outer component is parallel to the spinal axis, thereby further distracting the intervertebral disc space. The above-described process may be repeated with any number of spinal implant components until a desired height is reached. Although the present invention is not limited by any theory of operation, it is believed that the described device may improve the manner in which a disc space between vertebrae is expanded. Introducing pieces of a spinal implant incrementally as described herein may be preferable to an implant having a mechanical device for expanding which may be subject to a greater chance of failure. Further, the described implant and technique may diminish the amount of impact needed to insert the implant into the disc space.

One aspect of the present invention may provide a system for spinal fusion. The system according to this aspect of the invention may include a spinal implant and a positioning system. The spinal implant may define a longitudinal axis. The spinal implant may also include a plurality of spinal implant components. In this aspect, each implant component may be configured to nest inside an adjacent one of the plurality of spinal implant components when in an implanted position. Further in this aspect, the positioning system may be configured to insert each one of the plurality of spinal implant components into the implanted position. The positioning system may be configured to rotate the spinal implant to distract an intervertebral disc space.

According to another aspect of the invention, each of the plurality of spinal implant components may be a spacer having a first dimension in a first plane and a second dimension in a second plane. The first plane may be transverse to the second plane. The second dimension may be larger than the first dimension. The first and second planes may extend parallel to the longitudinal axis. The first plane may be orthogonal to the second plane. According to another aspect of the invention, each spacer may be configured to be inserted into the intervertebral disc space and rotated to distract the intervertebral disc space. According to another aspect of the invention, the positioning system may include a guidance structure configured to guide each spinal implant component into the implanted position. The guidance structure may be a shaft extending along the longitudinal axis. The shaft may be configured to receive each of the plurality of spinal components such that the spinal components are adapted to be translated along a length of the shaft. The shaft may include a proximal end adapted to receive a pushing unit. The shaft may include a distal end adapted to couple to the spinal implant.

According to yet another aspect of the invention, the positioning system may include a pushing unit configured to couple to the guidance structure and translate relative to the guidance structure. The pushing unit may be configured to push each one of the plurality of spinal components into the implanted position. The pushing unit may be configured to detachably couple to the guidance structure such that rotation of the pushing unit causes a simultaneous rotation of the shaft. In some aspects of the invention, the pushing unit may be a cannulated tube configured to extend and translate along the longitudinal axis. The cannulated tube may include a distal end configured to contact the spinal implant components and push the components into the implant position. The cannulated tube may further include a proximal end coupled to an actuation device.

According to yet another aspect of the invention, the positioning system may include an actuation device configured to rotate the spinal implant about the longitudinal axis. In some aspects, the actuation device may be a gripping tool adapted to extend along a second axis perpendicular to the longitudinal axis when the system is in an assembled condition. In some aspects, the gripping tool may be a handle coupled to a proximal end of the positioning system. The handle may be coupled to the proximal end of the positioning system in such a way that rotation of the handle causes a simultaneous rotation of the positioning system. The actuation device may be adapted to rotate the implant independently of a separate tool of the positioning system for inserting the spinal implant components into the implant position. In some aspects of the invention, the positioning system may include a plurality of pushing units. In some aspects, at least two of the pushing units may be configured to advance a different one of the spinal implant components into the implanted position.

Another aspect of the present invention provides a method for implanting a spinal implant. The method according to this aspect of the invention may include: positioning a first spinal implant component in an intervertebral disc space; rotating the first spinal implant component to distract the intervertebral disc space; positioning a second spinal implant component such that the second spinal implant component mates with the first spinal implant component; and rotating the first and second spinal implant components to distract the intervertebral disc space. In accordance with this aspect of the invention, the step of positioning the first spinal implant component may include coupling the first spinal implant component to a guidance structure. The first spinal implant component may have a first dimension and a second dimension larger than the first dimension. According to another aspect of the invention, the step of positioning the first spinal implant component may include advancing a pushing unit along a length of a guidance structure. The pushing unit may surround the guidance structure and be coupled to an actuation device.

According to another aspect of the invention, the positioning step may include orienting the first spinal implant component such that the first spinal implant component extends along a longitudinal axis. In this aspect, the second dimension of the first spinal implant component may be perpendicular to an axis of the spine. In another aspect of the invention, the step of rotating the first spinal implant component may include rotating the first spinal implant component about the longitudinal axis from an orientation in which the second dimension is transverse to the axis of the spine to an orientation in which the second dimension is parallel to the axis of the spine. According to yet another aspect of the invention, the rotating step may include rotating the first spinal implant component from an orientation in which the second dimension is perpendicular to the axis of the spine to an orientation is which the second dimension is parallel to the axis of the spine.

According to yet another aspect of the invention, the step of positioning the second spinal implant component may include retracting the pushing unit from the guidance structure, disposing the second spinal implant component around the guidance structure, and advancing the pushing unit along the length of the guidance structure such that the pushing unit contacts and advances the second spinal implant component along the length of the guidance structure and into the intervertebral disc space to mate with the first spinal implant component. In this aspect, the second spinal implant component may have a first dimension and second dimension larger than the first dimension. According to another aspect, the second implant component may extend along a longitudinal axis and the second implant component may be positioned such that the second dimension of the second implant is oriented in a direction transverse to an axis of the spine. According to yet another aspect of the invention, the step of rotating the second spinal implant component may include rotating the second spinal implant about the longitudinal axis from an orientation in which the second dimension of the second spinal implant component is transverse to the axis of the spine to an orientation in which the second dimension is parallel to the axis of the spine. According to yet another aspect, the second spinal implant component may mate with the first spinal implant component such that the second dimension of the first spinal implant component is aligned with the first dimension of the second spinal implant component.

In accordance with a further aspect of the invention, a spinal implant may include a first implant component and a second implant component. The first implant component may extend along a longitudinal axis, and the first implant component may define a first dimension disposed on a first plane and a second dimension disposed on a second place transverse to the first plane. The second dimension may be greater than the first dimension. In this aspect, the second implant component may extend along the longitudinal axis, and the second implant component may define a first dimension disposed on a first plane and a second dimension disposed on a second plane transverse to the first plane. The second dimension may be greater than the first dimension. In this aspect, the second implant component may be adapted to mate with the first implant component such that the first dimension of the second implant component aligns with the second dimension of the first implant component.

In another aspect of the invention, the first implant component may be configured to nest inside the second implant component such that an outer surface of the first implant component along the second dimension abuts an inner surface of the second implant component along the first dimension. According to yet another aspect of the invention, the first and second planes of the first implant component may be perpendicular to one another. Further in this aspect, the first and second planes of the second implant component may be perpendicular to one another. In yet another aspect, the spinal implant may include a third implant component. In this aspect, the third implant component may extend along the longitudinal axis. Further, the third implant component may define a first dimension disposed in a first plane and a second dimension disposed in a second plane transverse to the first plane. The second dimension may be greater than the first dimension. In another aspect, the third implant component may be adapted to mate with the second implant component such that the first dimension of the third implant component aligns with the second dimension of the second implant component. In yet another aspect, the second implant component may be configured to nest inside the third implant component such that an outer surface of the second implant component along the second dimension abuts an inner surface of the third implant component along the first dimension.

### BRIEF DESCRIPTION OF THE DRAWINGS

FIG. 1A is a perspective view of a spinal fusion system according to an embodiment of the disclosure.

FIG. 1B is an exploded view of the spinal fusion system of FIG. 1A.

FIG. 2A is a perspective view of a spinal implant of the spinal fusion system of FIGS. 1A-B.

FIGS. 2B-D are perspective views of implant components of the spinal implant of FIG. 2A

FIG. 2E is a perspective view of the implant component of FIG. 2B nested inside the implant component of FIG. 2C.

FIG. 3 is an exploded view of a positioning system of the spinal fusion system of FIGS. 1A-B.

FIG. 4A is a perspective view of a guidance structure coupled to a first spinal implant component of the spinal fusion system of FIGS. 1A-B.

FIG. 4B is a perspective view of a positioning system coupled to a first spinal implant component of the spinal fusion system of FIGS. 1A-B.

FIG. 5A is an exploded view of a positioning system and first and second spinal implant components of the spinal fusions system of FIGS. 1A-B.

FIG. 5B is a perspective view of a positioning system coupled to first and second spinal implant components of the spinal fusion system of FIGS. 1A-B.

FIG. 6A is an exploded view of a positioning system and first, second and third spinal implant components of the spinal fusion system of FIGS. 1A-B.

FIG. 6B is a perspective view of a positioning system coupled to first, second and third spinal implant components of the spinal fusion system of FIGS. 1A-B.

FIG. 6C is a perspective view of the positioning system and spinal implant components of FIG. 6B after a rotation of the positioning system.

### DETAILED DESCRIPTION

As used herein, the term “proximal,” when used in connection with a device, an implant or components of an implant, refers to the end of the device or implant closer to the user when the device is being used as intended. On the other hand, the term “distal,” when used in connection with a device, an implant or components of an implant, refers to the end of the device or implant farther away from the user when the device is being used as intended. As used herein, the terms “about,” “generally,” “approximately,” and “substantially” are intended to mean that slight deviations from absolute are included within the scope of the term so modified.

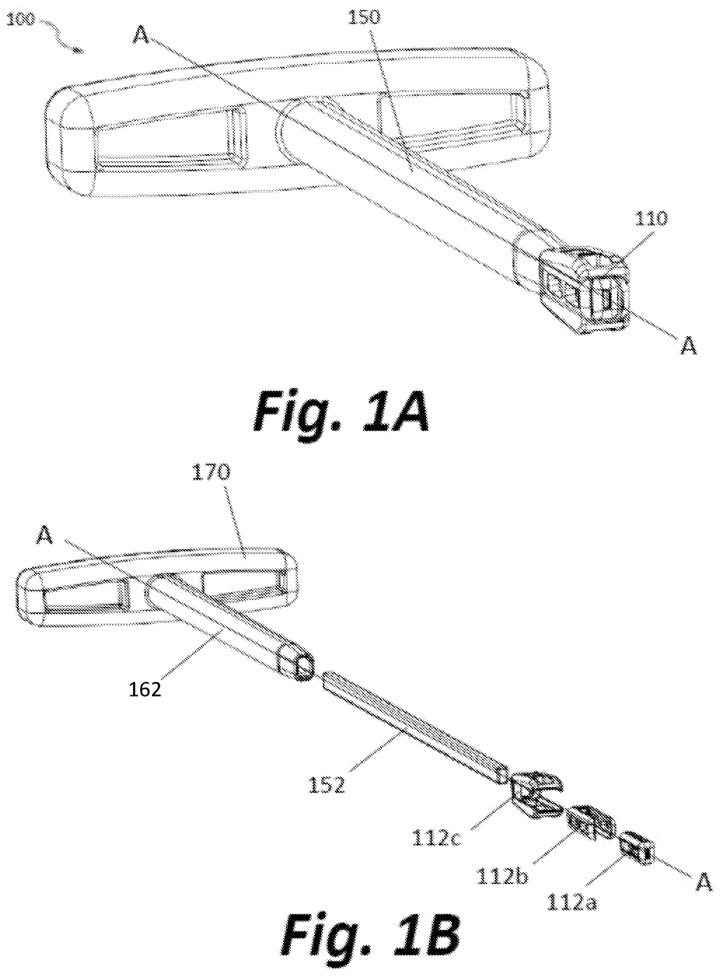

FIGS. 1A-B illustrate an embodiment of a spinal fusion system 100 for insertion of a spinal implant into an intervertebral disc space in accordance with the present disclosure. The spinal fusion system 100 is comprised of an augmentable spinal implant 110 and a positioning system 150 extending along longitudinal axis A as shown in FIG. 1A. The positioning system 150, which may include a guidance structure (such as shaft 152), a pushing unit (such as cannulated tube 160), and an actuation device (such as handle 170) as shown in FIG. 1B, may be manipulated by a surgeon or other operator to insert the spinal implant 110 into the intervertebral disc space. The spinal implant 110, which may include multiple components (such as first spacer 112a, second spacer 112b and third spacer 112c), may be manipulated to distract the disc space. The components of each of the implant 110 and the positioning system 150 along with the method of insertion will be described below in greater detail.

The augmentable spinal implant 110 may include a plurality of spinal implant components, and each component may vary in size. Each component may have a first side extending along a first plane defining a first dimension, and a second side extending along a second plane transverse to the first plane defining a second dimension. The second dimension may be larger than the first dimension. The second plane may be orthogonal to the first plane. The spinal implant 110 may have a first component sized to mate with and/or nest inside a second component such that the second dimension of the first component may align with the first dimension of the second component. Further, the second component may be sized to mate with and/or nest inside a third component such that the second dimension of the second component may align with the first dimension of the third component.

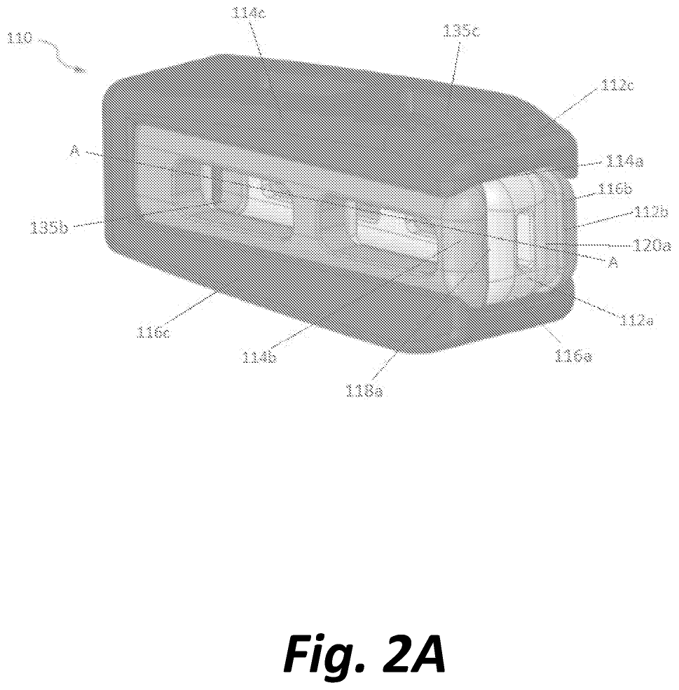

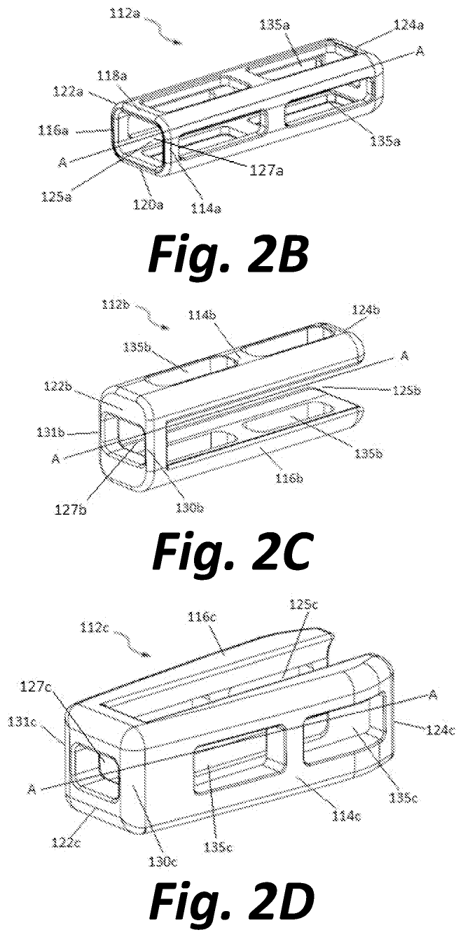

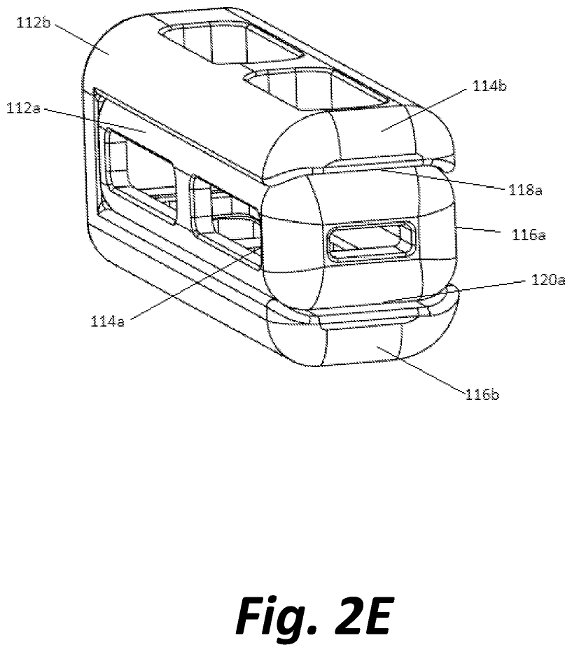

For example, in the embodiment illustrated in FIG. 2, the implant 110 includes a first implant component shown as first spacer 112a, a second implant component shown as second spacer 112b, and a third implant component shown as third spacer 112c. As illustrated in FIG. 2B, the first spacer 112a extends along a longitudinal axis A from a proximal end 122a to a distal end 124a. First spacer 112a has a first short side 114a substantially disposed on a first plane extending parallel to longitudinal axis A. The first spacer 112a further has a second short side 116a opposite the first short side 114a and substantially disposed on a second plane parallel to the first plane. The first spacer 112a further includes a first long side 118a disposed on a third plane transverse to the first and second planes, the third plane extending parallel to the longitudinal axis A. The third plane may be orthogonal to the first and second planes. The first spacer 112a further includes a second long side 120a disposed on a fourth plane transverse to the first and second planes, and parallel to the third plane. The width of short sides 114a, 116a (i.e., the distance along first short side 114a and second short side 116a, respectively, between first long side 118a and second long side 120a) defines a first dimension of the first spacer 112a. The width of long sides 118a, 120a (i.e. the distance along first long side 118a and second long side 120a, respectively, between first short side 114a and second short side 116a) defines a second dimension of the first spacer 112a. The second dimension is greater than the first dimension. That is, the width of the long sides 118a, 120a is greater than the width of the short sides 114a, 116a. In some examples, the first spacer 112a may measure approximately five millimeters in the first dimension (i.e., the width of the first and second short sides 114a, 116a), and the first spacer 112a may further measure approximately seven millimeters in the second dimension (i.e., the width of the first and second long sides 118a, 120a). The first and second short sides 114a, 116a and the first and second long sides 118a, 120a may define a channel 125a therebetween which is configured to receive a shaft of the positioning system 150. A bore 127a, which may have the same dimensions as the channel 125a along a plane perpendicular to longitudinal axis A, may be sized to receive the shaft 152 of the positioning system 150, such that rotation of the shaft 152 about longitudinal axis A induces corresponding rotation of the first spacer 112a about that axis. Further, the short sides 114a, 116a and long sides 118a, 120a define apertures 135a. The apertures on the first spacer 112a may communicate with apertures on one or more implant components (e.g., spacers of the implant 110) surrounding the first spacer 112a, as discussed below.

The spinal implant 110 further includes a second spacer 112b illustrated in FIG. 2C extending along longitudinal axis A from proximal end 122b to distal end 124b. The second spacer 112b has a first wing 114b extending from a first side of proximal end 122b, the first wing 114b substantially disposed on a first plane extending parallel to longitudinal axis A. Second spacer 114b further has a second wing 116b extending from a second side of proximal end 122b opposite the first side, the second wing 116b substantially disposed on a second plane parallel to and opposite the first plane. The first and second wings 114b, 116b define apertures 135b thereon. The width of first and second wings 114b, 116b (i.e., the distance along first wing 114b and second wing 116b, respectively, between first long edge 130b and second long edge 131b) defines a first dimension of the second spacer 112b.

The first and second wings 114b, 116b may each have an inner surface that may abut a surface of an implant component disposed inside the second spacer 112b when the implant 110 is in an assembled configuration. For example, the inner surface of the first wing 114b may abut the first long side 118a of the first spacer 112a, and the inner surface of the second wing 116b may abut the second long side 120a of the first spacer. Further, the first and second wings 114b, 116b may have an outer surface opposite the inner surface, such that the outer surfaces of each wing may face away from the implant component disposed inside the second spacer 112b. The distance between the outer surface of the first wing 114b and the outer surface of the second wing 116b defines a second dimension of the second spacer 112b. The second dimension is greater than the first dimension. That is, the distance between the outer surfaces of the first and second wings 114b, 116b is greater than the width of the wings 114b, 116b. In some examples, the second spacer 112b may measure approximately seven millimeters in the first dimension (i.e., the width of each wing 114b, 116b), and the second spacer 112b may measure approximately nine millimeters in the second dimension (i.e., the distance between the outer opposing surfaces of the first wing 114b and the second wing 116b).

The second spacer 112b defines a channel 125b therethrough, the channel 125b being generally rectangular-shaped and surrounded by the first wing 114b on the first side and the second wing 116b on the second side, and the channel being open on the third and fourth sides. The channel 125b may receive at least a portion of the positioning system 150 through a bore 127b on the proximal end 122b of the second spacer 112b. For example, the bore 127b may be sized to receive the shaft 152 of the positioning system 150 therethrough, so that rotation of the shaft 152 about longitudinal axis A induces corresponding rotation of the second spacer 112b about that axis. In that regard, the bore 127b of second spacer 112b may have the same dimensions along a plane perpendicular to the longitudinal axis A as the bore 127a of the first spacer 112a. The channel 125b is open on the distal end 124b of the second spacer 112b. The apertures 135b and/or open sides of the channel 125b of the second spacer 112b may communicate with apertures and/or open sides of a channel on implant components (e.g., spacers of the implant 110) that are disposed inside the second spacer 112b and/or surrounding the second spacer 112b.

The spinal implant 110 further includes a third spacer 112c illustrated in FIG. 2D extending along longitudinal axis A from proximal end 122c to distal end 124c. Third spacer 112c has a first wing 114c extending from a first side of proximal end 122b, the first wing 114b substantially disposed on a first plane extending parallel to longitudinal axis A. The third spacer 114c further has a second wing 116c extending from a second side of proximal end 122c opposite the first side, the second wing 116c substantially disposed on a second plane parallel to and opposite the first plane. The first and second wings 114c, 116c define apertures 135c thereon. The width of first and second wings 114c, 116c (i.e., the distance along first wing 114c and second wing 116c, respectively, between first long edge 130c and second long edge 131c) defines a first dimension of the third spacer 112c.

The first and second wings 114c, 116c may each have an inner surface that may abut a surface of an implant component disposed inside the third spacer 112c when the implant 110 is in an assembled configuration. For example, the inner surface of the first wing 114c may abut the first short side 114a of the first spacer 112a surrounded by the wings 114b, 116b of second spacer 112b, and the inner surface of the second wing 116b may abut the second short side 120a of the first spacer 112a surrounded by the wings 114b, 116b of second spacer 112b. Further, the first and second wings 114c, 116c of the third spacer 112c may have an outer surface opposite the inner surface, such that the outer surfaces of each wing may face away from the implant component(s) disposed inside the third spacer 112c. The distance between the outer surface of the first wing 114c and the outer surface of the second wing 116c defines a second dimension of the third spacer 112c. The second dimension is greater than the first dimension. That is, the distance between the outer surfaces of the first and second wings 114c, 116c is greater than the width of the wings 114c, 116c. In some examples, the third spacer 112c may measure approximately nine millimeters in the first dimension (i.e., the width of each wing 114c, 116c), and the third spacer 112c may measure approximately eleven millimeters in the second dimension (i.e., the distance between the outer opposing surfaces of the first wing 114c and the second wing 116c).

The third spacer 112c defines a channel 125c therethrough, the channel 125c being generally rectangular-shaped and surrounded by the first wing 114c on the first side and the second wing 116c on the second side, and the channel being open on the third and fourth sides. The channel 125c may receive at least a portion of the positioning system 150 through a bore 127c on the proximal end 122c of the third spacer 112c. For example, the bore 127c may be sized to receive the shaft 152 of the positioning system 150 therethrough, so that rotation of the shaft 152 about longitudinal axis A induces corresponding rotation of the third spacer 112c about that axis. In that regard, the bore 127c of the third spacer 112c may have the same dimensions along a plane perpendicular to the longitudinal axis A as the bores 127a, 127b of the respective first and second spacers 112a, 112b. The channel 125c is open on the distal end 124c of the third spacer 112c. The apertures 135c and/or open sides of the channel 125c of the third spacer 112c may communicate with apertures and/or open sides of a channel on implant components (e.g., spacers of the implant 110) that are disposed inside the third spacer 112c and/or surrounding the third spacer 112c.

The first spacer 112a, second spacer 112b and third spacer 112c are configured to mate with each other to form spinal implant 110. As shown in FIG. 2E, the first spacer 112a may be nested in the channel 125b of the second spacer 112b such that the first spacer 112a is positioned between the first and second wings 114b, 116b of the second spacer 112b. The first dimension of the second spacer 112b (i.e., the width of the wings 114b, 116b) is approximately equal to the second dimension of the first spacer 112a (i.e., the width of the first and second long sides 118a, 120a). Thus, the first wing 114b of the second spacer 112b substantially abuts and aligns with the first long side 118a of the first spacer 112a, and the second wing 116b of the second spacer 112b substantially abuts and aligns with the second long side 120a of the first spacer 112a. The open sides of the channel 125b of the second spacer 112b are generally filled by the short surfaces 114a, 116a of the first spacer 112a.

As further illustrated in FIG. 2A, the first and second spacers 112a, 112b may be nested inside the third spacer 112c such that the first and second spacers 112a, 112b are positioned between the first and second wings 114c, 116c of the third spacer 112c. The first dimension of the third spacer 112c (i.e., the width of the wings 114c, 116c of the third spacer 112c) is approximately equal to the second dimension of the second spacer 112b (i.e., the distance between the outer surfaces of the wings 114b, 116b of the second spacer 112b). Thus, the first wing 114c of the third spacer 112c substantially abuts and aligns with the first short surface 114a of the first spacer 112a which is disposed in the open third side of the channel 125b of the second spacer 112b. The second wing 116c of the third spacer 112c substantially abuts and aligns with the second short surface 116a of the first spacer 112a which is disposed in the open fourth side of the channel 125b of the second spacer 112b. Each of the spacers may include a means for mating on the outer surface of an inner component and/or the inner surface of an outer component such as a track or the like to facilitate a secure attachment between the spinal implant components. It should be noted that although only three spacers are shown in the illustrated embodiment, it is contemplated that any number of spacers may be used to achieve a desired size of the implant.

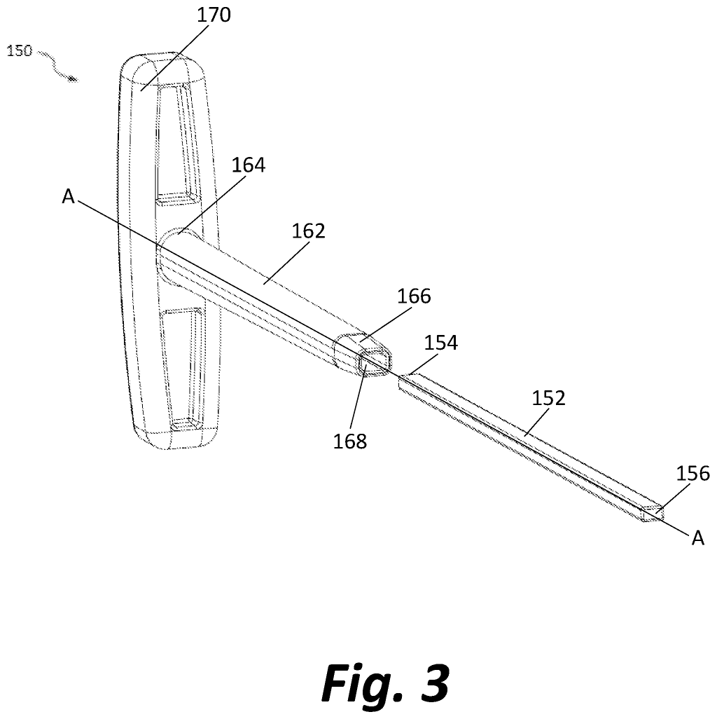

Also included in the spinal fusion system 100 is a positioning system 150 as shown in FIG. 3. The positioning system 150 may be configured to insert the implant 110 into the implanted position. The positioning system 150 may additionally (or alternatively) be configured to rotate the implant 110 to distract the intervertebral disc space. The positioning system 150 may include a guidance structure (e.g., shaft 152) configured to guide each spinal implant component (e.g., first spacer 112a, second spacer 112b and third spacer 112c) into the implanted position within the intervertebral disc space. The shaft 152 extends from a proximal end 154 to a distal end 156 along longitudinal axis A and is configured to receive each of the spacers 112a-c such that the spacers 112a-c may surround the shaft 152 and translate along the longitudinal axis A relative to the shaft 152, as described below in greater detail. The distal end 156 of the shaft 152 is configured to couple to the implant 110 by extending through the channel 125a of the first spacer 112a to facilitate insertion of the implant 110 into the implanted position.

The positioning system 150 may include a pushing unit which may be received by the guidance structure to contact an implant component disposed around the guidance structure. For example, the pushing unit may be a cannulated tube 162 as shown in FIG. 3. The cannulated tube 162 extends from a proximal end 164 to a distal end 166 along longitudinal axis A when in an assembled configuration. The cannulated tube 162 may be configured to detachably couple to the shaft 152 and translate along the longitudinal axis A relative to the shaft 152. For example, the shaft 152 may have a rectangular cross-section and the cannulated tube 162 may define a central lumen 168, the central lumen 168 having a similarly sized rectangular cross-section configured to receive the shaft 152 such that the cannulated tube 162 may be freely translated proximally and distally along the longitudinal axis A relative the shaft 152. The rectangular cross-sections of the shaft 152 and the central lumen 168 of the cannulated tube 162 may form a connection between the shaft 152 and the cannulated tube 162 such that a rotation of the cannulated tube 162 may cause a simultaneous rotation of the shaft 152. It is contemplated that the shaft 152 and cannulated tube 162 may have cross-sections of any shape that would promote a similar connection such that the shaft 152 and cannulated tube 162 may rotate in sync and/or translate relative to one another. As a spinal implant component, such as one of spacers 112a-c, is disposed around the shaft 152, the distal end 166 of the cannulated tube 162 may be translated over the shaft 152 and may contact the proximal end of the spinal implant component to advance the implant component distally into the implanted position.

The positioning system 150 may further include an actuation device which may be manipulated by an operator or user to rotate the spinal implant 110. Such rotation may involve rotating the positioning system 150 connected to the spinal implant 110. The actuation device may be a gripping tool such as a handle 170 as illustrated in FIG. 3. The handle 170 may be configured to couple to the proximal end 164 of the cannulated tube 162. The handle 170 extends in a direction perpendicular to the longitudinal axis A, so as to allow a user or operator to apply a torque to the handle 170 to cause rotation of the positioning system 150. That is, rotation of the handle 170 about the longitudinal axis A causes rotation of the cannulated tube 162 about the longitudinal axis A, which further causes a rotation of the shaft 152 about the longitudinal axis A, which may further cause a rotation of the spinal implant 110 coupled thereto about the longitudinal axis A. The handle 170 may be rotated in either a clockwise or a counter-clockwise direction.

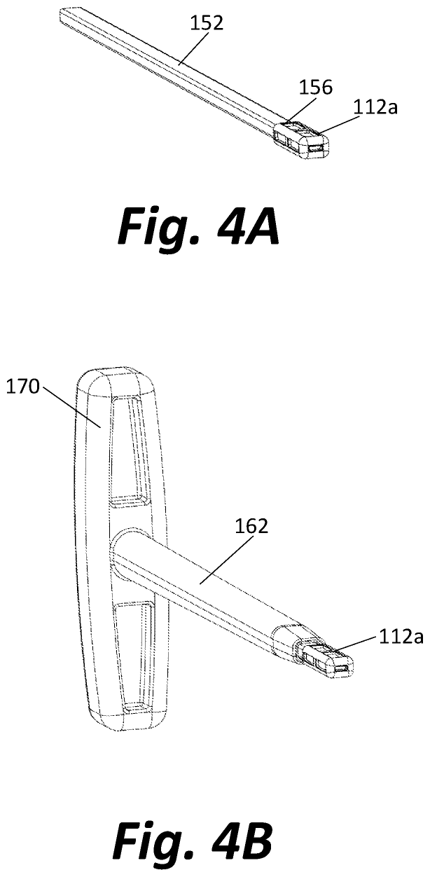

A method of implanting the spinal implant 110 is described herein. The method may include positioning a first spinal implant component (e.g., the first spacer 112a) within an intervertebral disc space. Positioning the spacer 112a may include coupling the spacer 112a to the positioning system 150 to allow a user or operator to manipulate the first spacer 112a within the intervertebral disc space and/or insert the first spacer 112a into the intervertebral disc space. For example, the first spacer 112a may be coupled to the distal end 156 of the shaft 152 as shown in FIG. 4A. Illustrated in FIG. 4B, the cannulated tube 162 may be inserted over the shaft 152 to operate the positioning system 150 to maneuver the first spacer 112a into the disc space. As described above, the first spacer 112a may have a shape in which the first spacer 112a extends in a first dimension and a second dimension, and the second dimension is greater than the first dimension. In some examples, the first spacer 112a may measure approximately five millimeters in the first dimension and approximately seven millimeters in the second dimension.

The first spacer 112a may be first inserted into the intervertebral disc space such that the second dimension is oriented transverse to an axis of the spine. For example, the first spacer 112a may be oriented such that the surrounding vertebrae are separated by the width of the first dimension of the first spacer 112a, e.g., five millimeters. After the first spacer 112a is placed in the implanted position (i.e., in the intervertebral disc space), the first spacer 112a may be rotated to reorient the first spacer 112a so that the second dimension of the first spacer 112a is substantially parallel to the axis of the spine, thereby distracting the intervertebral disc space. For example, the first spacer 112a may be rotated by rotating the handle 170 coupled to the cannulated tube 162 to thereby rotate the shaft 152 coupled to the first spacer 112a. In some examples, the first spacer 112a may be inserted into the implanted position in an orientation in which the second dimension is substantially perpendicular to the axis of the spine, and the first spacer 112a may be rotated approximately ninety degrees to orient the second dimension substantially parallel to the axis of the spine causing a distraction of the intervertebral disc space. In an example in which the second dimension of the first spacer 112a measures approximately seven millimeters and the first dimension of the first spacer 112a measures approximately five millimeters, the intervertebral space may be distracted approximately two millimeters upon rotation of the first spacer 112a. After the first spacer 112a is implanted and rotated to distract the intervertebral space, the cannulated tube 162 may be retracted by translating it proximally relative to the shaft 152 so that the shaft 152 remains coupled to the implanted first spacer 112a while the pushing unit 160 is removed. The shaft 152 may remain coupled to the first spacer 112a while an additional spinal implant component is prepared for implantation as described below. Alternatively, if a desired height of the intervertebral disc space has been reached upon rotation of the first spacer 112a, the shaft 152 may be decoupled from the first spacer 112a.

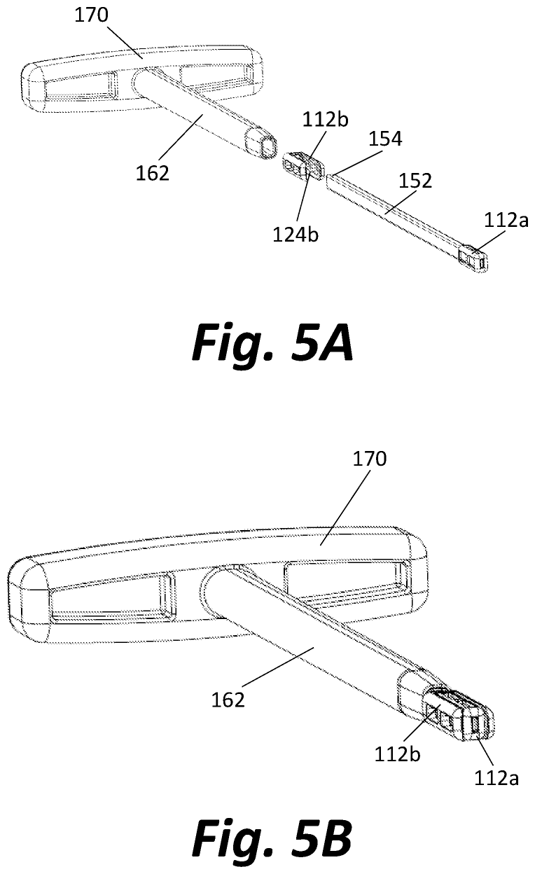

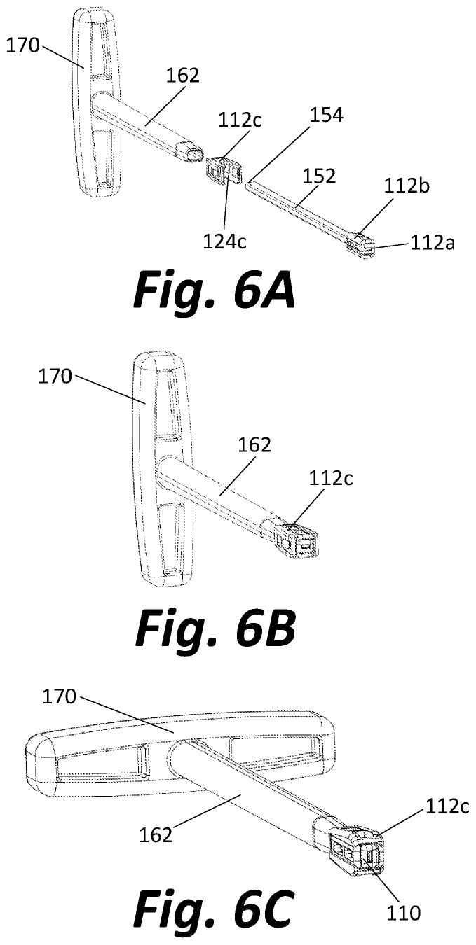

The method of implanting the spinal implant 110 may further include positioning a second spinal implant component into the intervertebral disc space and mating the second implant component with the first implant component in the implanted position. For example, after the first spacer 112a is implanted and the pushing unit 160 is decoupled from the shaft 152 as described above, a second spacer 112b may be coupled to the shaft 152. That is, the second spacer 112b may be inserted over the proximal end 154 of the shaft 152 such that the shaft 152 is received by the channel 125b of the second spacer 112b, as shown in FIG. 5A. The second spacer 112b may be sized so that the channel 125b through which the shaft 152 is received may provide a stable connection between the spacer 112b and the shaft 152, and the spacer 112b may freely translate in the distal direction along the longitudinal axis A relative to the shaft 152. After the spacer 112b is disposed over the proximal end 154 of the shaft 152, the cannulated tube 162 may be inserted over the proximal end 154 of the shaft 152 to contact the second spacer 112b and advance the second spacer 112b distally along the shaft 152.

The second spacer 112b may be disposed on the shaft 152 in an orientation that may promote mating between the first spacer 112a and the second spacer 112b in the implanted position. For example, as described above, the second spacer 112b may define a first dimension and a second dimension larger than the first dimension. The first dimension of the second spacer 112b may be approximately equal to the second dimension of the first spacer 112a. For example, the first spacer 112a may measure approximately seven millimeters in the second dimension and the second spacer 112 may measure approximately seven millimeters in the first dimension. That is, as described above, the first wing 114b of the second spacer 112b may substantially cover and be disposed on the first long side 118a of the first spacer 112a. Further, the second wing 116b of the second spacer 112b may substantially cover and be disposed on the second long side 120a of the first spacer 112a. The second spacer 112b may be mated to the first spacer 112a such that rotation of the first spacer 112a may cause simultaneous rotation of the second spacer 112b. The second spacer 112b may be oriented such that the second dimension of the second spacer 112b may be transverse to the axis of the spine as the second spacer 112b is advanced into the intervertebral space to be received by the first spacer 112a.

After the second spacer 112b is in the implanted position and mated with the first spacer 112a, the spinal implant 110 may be rotated to further distract the intervertebral space. For example, the handle 170 may be rotated by the user or operator, which may simultaneously rotate the cannulated tube 162, the shaft 152, the first spacer 112a and the second spacer 112b. The rotation of the spinal implant 110 may orient the second spacer 112b such that the second dimension of the second spacer 112b is parallel to the axis of the spine. In some examples, the second spacer 112b may be inserted into the intervertebral space in an orientation in which the second dimension is substantially perpendicular to the axis of the spine, and the second spacer 112b may be rotated approximately ninety degrees such that the second dimension is substantially parallel to the axis of the spine. In an example in which the second spacer 112b measures approximately seven millimeters in the first dimension and nine millimeters in the second dimension, the rotation of the second spacer 112b may cause a distraction of approximately two millimeters in the intervertebral disc space. After the second spacer 112b is implanted and rotated to distract the intervertebral space, the cannulated tube 162 may be retracted by translating it proximally relative to the shaft 152 so that the shaft 152 remains coupled to the implanted first spacer 112a of the spinal implant 110 while the cannulated tube 162 is removed. The shaft 152 may remain coupled to the first spacer 112a while an additional spinal implant component is prepared for implantation. Alternatively, if a desired height of the intervertebral disc space has been reached upon rotation of the second spacer 112b, the shaft 152 may be decoupled from the first spacer 112a.

The method of implanting the spinal implant 110 may further include positioning a third spinal implant component into the intervertebral disc space and mating the third implant component with the second and first implant components in the implanted position. The method of inserting a third implant component may be substantially similar to the method for inserting a second implant component described above. For example, after the second spacer 112b is implanted and mated to the first spacer 112a and the cannulated tube 162 is decoupled from the shaft 152 as described above, a third spacer 112c may be coupled to the shaft 152. The third spacer 112c may be inserted over the proximal end 154 of the shaft 152 such that the shaft 152 is received by the channel 125c of the third spacer 112c. The third spacer 112c may be sized so that the channel 125c through which the shaft 152 is received may provide a stable connection between the third spacer 112c and the shaft 152, and the third spacer 112c may freely translate in the distal direction along the longitudinal axis A relative to the shaft 152. After the third spacer 112c is disposed over the proximal end 154 of the shaft 152, the cannulated tube 162 may be inserted over the proximal end 154 of the shaft 152 to contact the third spacer 112c and advance the third spacer 112c distally along the shaft 152.

The third spacer 112c may be disposed on the shaft 152 in an orientation that may promote mating between the third spacer 112c and the first and second spacers 112a-b in the implanted position. For example, as described above, the third spacer 112c may define a first dimension and a second dimension larger than the first dimension. The first dimension of the third spacer 112c may be approximately equal to the second dimension of the second spacer 112b. For example, the second spacer 112b may measure approximately nine millimeters in the second dimension and the third spacer 112c may measure approximately nine millimeters in the first dimension. That is, as described above, the first wing 114c of the third spacer 112c may substantially cover and be disposed on the first short surface 114a of the first spacer 112a surrounded by the wings 114b, 116b of the second spacer 112b. Further, the second wing 116c of the third spacer 112c may substantially cover and be disposed on the second short surface 116a of the first spacer 112a surrounded by the wings 114b, 116b of the second spacer 112b. The third spacer 112c may be mated to the first and second spacers 112a-b such that rotation of the first spacer 112a may cause simultaneous rotation of the second and third spacers 112b-c. The third spacer 112c may be oriented such that the second dimension of the third spacer 112c may be transverse to the axis of the spine as the third spacer 112c is advanced into the intervertebral space to be received by the first and second spacers 112a-b.

After the third spacer 112c is in the implanted position and mated with the first and second spacers 112a-b, the spinal implant 110 may be rotated to further distract the intervertebral space. For example, the handle 170 may be rotated by the user or operator, which may simultaneously rotate the cannulated tube 162, the shaft 152, and the spinal implant 110 formed by the mated first, second and third spacers 112a-c. The rotation of the spinal implant 110 may orient the third spacer 112c such that the second dimension of the third spacer 112c is parallel to the axis of the spine. In some examples, the third spacer 112c may be inserted into the intervertebral space in an orientation in which the second dimension is substantially perpendicular to the axis of the spine, and the third spacer 112c may be rotated approximately ninety degrees such that the second dimension is substantially parallel to the axis of the spine. In an example in which the third spacer 112c measures approximately nine millimeters in the first dimension and approximately eleven millimeters in the second dimension, the rotation of the third spacer 112c may distract the intervertebral disc space by two millimeters. After the third spacer 112c is implanted and rotated to distract the intervertebral space, the cannulated tube 162 may be retracted by translating it proximally relative to the shaft 152 so that the shaft 152 remains coupled to the implanted first spacer 112a of the spinal implant 110 while the cannulated tube 162 is removed. The shaft 152 may remain coupled to the first spacer 112a while an additional spinal implant component is prepared for implantation. Alternatively, if a desired height of the intervertebral disc space has been reached upon rotation of the third spacer 112c, the shaft 152 may be decoupled from the first spacer 112a. It should be noted that the above-described method may continue with any number of spinal implant components in any size until the intervertebral space is distracted by a desired amount. It should also be noted that after the spinal implant is in the implanted position in the intervertebral disc space, the space within and/or surrounding the implant may be packed with material to promote fusion of the vertebrae, such as autologous and/or allogenic bone graft, a bone growth enabling matrix, and/or bone growth stimulating substances. For example, such fusion promoting material may be positioned within the interior channels 124a-c of the respective spacers 112a-c, such that the fusion promoting material communicates with the intervertebral space via the apertures 135a-c in the spacers. Further, the spinal implant may be secured in place with anchoring members, such as pedicle screws, spinal rods and the like or any other stabilizing method to facilitate fusion of the harvested bone with the surrounding vertebrae.

Although the positioning system 150 has been shown herein as comprising multiple subcomponents that operate in conjunction with one another in order to perform various positioning functions, such as advancing/guiding the spinal implant components into the intervertebral disc space and rotating the implant components, alternative positioning systems (not shown) in accordance with the present invention may include separate components that independently perform at least some of such functions. For example, the pushing unit (e.g., cannulated tube 162) need not be coupled to the actuation device (e.g., handle 170) for rotating the implant components. Instead, the actuation device may be engaged directly with the guidance structure (e.g., shaft 152) or the implant 110 (with or without the pushing device in place) in order to rotate the implant within the intervertebral disc space. It is also contemplated that more than one pushing unit may be included for advancing different implant components. For example, there may be a separate pushing unit (e.g., cannulated tube 160 or other pushing device) for each implant component (e.g., one pushing unit for the first spacer 112a and a separate pushing unit for the second spacer 112b).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for spinal fusion comprising:
a spinal implant defining a longitudinal axis, the spinal implant including a plurality of spinal implant components, each component configured to nest inside an adjacent one of the plurality of spinal implant components in an implanted position; and
a positioning system configured to insert each one of the plurality of spinal implant components into the implanted position,
wherein the positioning system is configured to rotate the spinal implant to distract an intervertebral disc space.

2. The system of claim 1, wherein each of the plurality of spinal implant components is a spacer having a first dimension in a first plane and a second dimension in a second plane transverse to the first plane, the second dimension larger than the first dimension.

3. The system of the claim 2, wherein the first and second planes extend parallel to the longitudinal axis, and the first plane is orthogonal to the second plane.

4. The system of claim 2, wherein each spacer is configured to be inserted into the intervertebral disc space and rotated to distract the intervertebral disc space.

5. The system of claim 1, wherein the positioning system includes a guidance structure configured to guide each spinal implant component into the implanted position.

6. The system of claim 5, wherein the guidance structure is a shaft extending along the longitudinal axis, the shaft configured to receive each of the plurality of spinal components such that the spinal components are adapted to be translated along a length of the shaft.

7. The system of claim 6, wherein the shaft includes a proximal end adapted to receive a pushing unit, and the shaft includes a distal end adapted to couple to the spinal implant.

8. The system of claim 5, wherein the positioning system includes a pushing unit configured to couple to the guidance structure and translate relative to the guidance structure.

9. The system of claim 8, wherein the pushing unit is configured to push each one of the plurality of spinal components into the implanted position.

10. The system of claim 8, wherein the pushing unit is configured to detachably couple to the guidance structure such that a rotation of the pushing unit causes a simultaneous rotation of the shaft.

11. The system of claim 8, wherein the pushing unit is a cannulated tube configured to extend and translate along the longitudinal axis.

12. The system of claim 11, wherein the cannulated tube includes a distal end configured to contact the spinal implant components and push the components into the implanted position, and the cannulated tube includes a proximal end coupled to an actuation device.

13. The system of claim 1, wherein the positioning system includes an actuation device configured to rotate the spinal implant about the longitudinal axis.

14. The system of claim 13, wherein the actuation device is a gripping tool adapted to extend along a second axis perpendicular to the longitudinal axis when the system is in an assembled condition.

15. The system of claim 14, wherein the gripping tool is a handle coupled to a proximal end of the positioning system such that a rotation of the handle causes a simultaneous rotation of the positioning system.

16. The system of claim 13, wherein the actuation device is adapted to rotate the implant independently of a separate tool of the positioning system for inserting the spinal implant components into the implanted position.

17. The system of claim 1, wherein the positioning system includes a plurality of pushing units, at least two of the pushing units configured to advance a different one of the spinal implant components into the implanted position.

18. A method for implanting a spinal implant comprising:
positioning a first spinal implant component in an intervertebral disc space;
rotating the first spinal implant component to distract the intervertebral disc space;
positioning a second spinal implant component such that the second spinal implant component mates with the first spinal implant component; and
rotating the first and second spinal implant components to distract the intervertebral disc space.

19. The method of claim 18, wherein the step of positioning the first spinal implant component includes coupling the first spinal implant component to a guidance structure, the first spinal implant component having a first dimension and a second dimension larger than the first dimension.

20. The method of claim 19, wherein the step of positioning the first spinal implant component includes advancing a pushing unit along a length of the guidance structure, the pushing unit surrounding the guidance structure and coupled to an actuation device.

21-33. (canceled)
